(19) United States
(12) Patent Application Publication
(10) Pub. No.: US 2014/0088451 A1
(43) Pub. Date: Mar. 27, 2014

(54) SYSTEM AND METHOD FOR MANAGING ECG ACQUISITION DEVICES

(71) Applicants: Matthew Pemberton, West Bend, WI (US); Brian Young, Germantown, WI (US)

(72) Inventors: Matthew Pemberton, West Bend, WI (US); Brian Young, Germantown, WI (US)

(73) Assignee: GENERAL ELECTRIC COMPANY, Schenectady, NY (US)

(21) Appl. No.: 13/627,263

(22) Filed: Sep. 26, 2012

(51) Int. Cl. A61B 5/044; A61B 5/0402

(52) U.S. Cl. 600/523; 600/509

(57) ABSTRACT

An ECG device management system is disclosed herein. The ECG device management system includes a display, and a processor. The processor is configured to receive data from a plurality of ECG acquisition devices. The processor is further configured to process the data and to generate an output on the display based on the processed data. The output is adapted to facilitate the optimization of the ECG acquisition devices performance.

### FIELD OF THE INVENTION

This disclosure relates generally to a system and method for managing ECG acquisition devices.

### BACKGROUND OF THE INVENTION

An electrocardiograph is a cardiac diagnostic/monitoring system adapted to record the electrical activity of a patient's heart. The electrocardiograph generally includes an array of sensors or transducers placed at predetermined positions on a patient's body. The recorded data from an electrocardiograph is generally displayed in the form of a graph that is often referred to as an electrocardiogram (ECG).

Hospitals and clinics commonly implement multiple electrocardiograph devices to monitor and diagnose patients, which may include different diagnostic applications such as resting ECG, stress testing ECG, and cardiac defibrillators. As an example, large multi-facility hospitals frequently deploy many electrocardiograph devices disposed in different facilities while maintaining a central domain of ownership and control. One problem with such an arrangement is that it is difficult to manage and optimize the performance of these electrocardiograph devices.

### BRIEF DESCRIPTION OF THE INVENTION

The above-mentioned shortcomings, disadvantages and problems are addressed herein which will be understood by reading and understanding the following specification.

In an embodiment, an ECG device management system includes a display and a processor. The processor is configured to receive data from a plurality of ECG acquisition devices. The processor is further configured to process the data and to generate an output on the display based on the processed data. The output is adapted to facilitate the optimization of the ECG acquisition devices performance.

In another embodiment, an ECG device management system includes a processor configured to receive data from a plurality of ECG acquisition devices, an algorithm configured to identify patterns within the received data, and a display operatively connected to the processor. The display is configured to visually convey output related to the identified patterns. The output is adapted to facilitate the optimization of the ECG acquisition devices performance.

In another embodiment, a method includes receiving data from a plurality of ECG acquisition devices, and implementing a processor to analyze the data. The method also includes generating a recommendation based on the analyzed data. The recommendation is adapted to facilitate the optimization of the ECG acquisition devices performance.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of an ECG acquisition device operatively connected to a patient via a twelve lead system in accordance with an embodiment;

FIG. 2 is a schematic illustration of an ECG device management system operatively connected to a plurality of facilities comprising ECG acquisition devices in accordance with an embodiment; and

FIG. 3 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

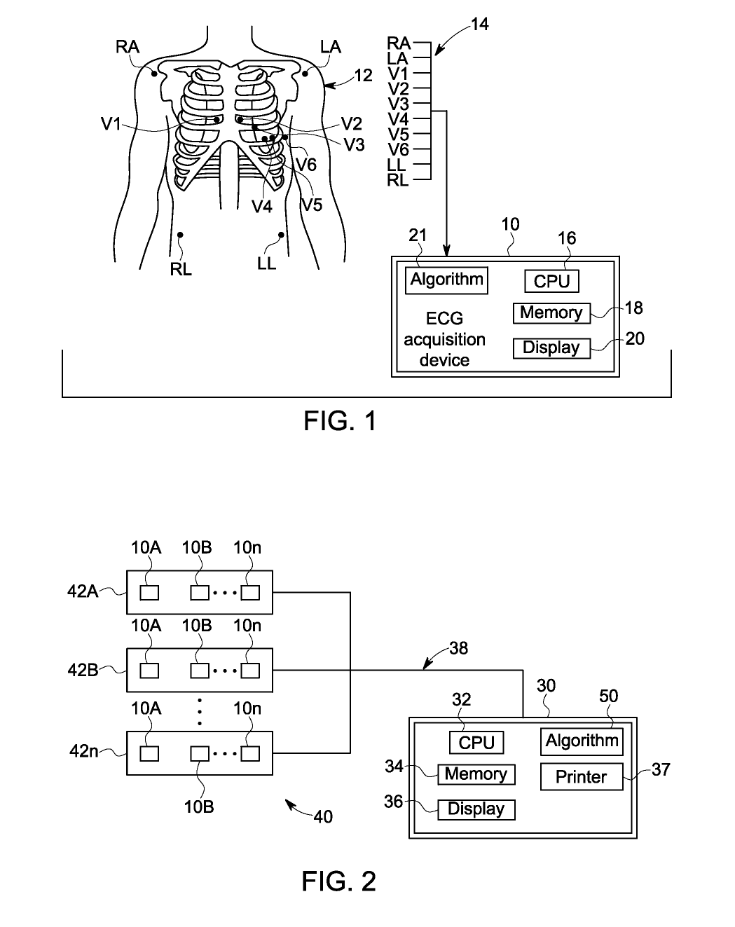

Referring to FIG. 1, a schematically represented exemplary electrocardiogram (ECG) acquisition device 10 is shown in accordance with an embodiment. The ECG acquisition device 10 is adapted to measure an electrical signal generated by a patient's heart.

The ECG acquisition device 10 can be coupled to a patient 12 by an array of sensors or transducers 14. In the illustrated embodiment, the array of sensors 14 include a right arm electrode RA; a left arm electrode LA; chest electrodes V1, V2, V3, V4, V5 and V6; a right leg electrode RL; and a left electrode leg LL for acquiring a standard twelve lead, ten-electrode electrocardiogram (ECG) signal. The twelve ECG leads include leads I, II, V1, V2, V3, V4, V5 and V6 which can be acquired directly from the patient leads, and leads III, aVR, aVL and aVF which can be derived using Einthoven's law. In other embodiments, alternative configurations of sensors and sensor locations can be used to acquire a standard or non-standard ECG signal. Alternatively other ECG acquisition devices and configurations may be implemented.

The ECG acquisition device 10 may comprise a central processing unit (CPU) 16 a memory device 18 a display 20 and an algorithm 21. The CPU 16 may receive ECG data from the sensors 14, and process or analyze the ECG data (e.g., based on instructions from the algorithm 21) in any known manner to facilitate patient diagnosis. The CPU 16 may transmit the processed ECG data to the memory device 18 for storage, and/or the display 20 for communication to a user. The processed data from the CPU 16 is commonly represented by the display 20 in a graphical form referred to as an ECG.

The CPU 16 is further configured to generate location data based on the location of the ECG acquisition device 10; quality data based on the quality of the ECG data; usage data based the frequency of ECG acquisition device usage; and identification data based on the identity of a specific ECG acquisition device. The CPU 16 can calculate location data in accordance with one embodiment wherein the ECG acquisition device 10 is part of a network by identifying its node or network connection point. As the physical location of each node within a network is known, it can be assumed that an ECG acquisition device 10 connecting to the network through a given node must be in relative close proximity. Alternatively, the CPU 16 may leverage GPS or any other known technology for generating location data.

The CPU 16 can calculate quality data can based on ECG file size data. It has been established that ECG data files stored in a lossless compression format that are greater than approximately 35 KB, and ECG data files stored in a lossy compression format that are greater than approximately 17 KB, are likely of poor quality. Lossless data compression and lossy data compression are well known to those skilled in the art and will therefore not be described in detail. This poor quality may, for example, be attributable to faulty sensor 14 leadwires, one or more disconnected sensors 14, or an inexperienced technician. Alternatively the CPU 16 can calculate quality data based on an assessment of sensor signal strength, or any other known method for assessing ECG data quality.

The CPU 16 can generate usage data based on the volume of ECG data acquired and/or transmitted. The CPU 16 can generate identification data based on the serial number or any other identifier of the associated ECG acquisition device 10.

Referring to FIG. 2, an ECG device management system 30 is shown in accordance with an embodiment. The management system 30 may comprise a CPU 32, a memory 34 a display 36, a printer 37 and an algorithm 50. The display 36 may comprise known technology to visually or audibly convey media and may according to one embodiment comprise a monitor. The ECG device management system 30 will be described for illustrative purposes as being operatively connected via a network 38 with a multi facility hospital 40 comprising a plurality of geographically disparate facilities 42A-42N. It should be appreciated that the ECG device management system 30 is scalable to accommodate a variety of different hospital system configurations and sizes.

The facilities 42A-42N each comprise a plurality of ECG acquisition devices 10A-10N that are generally similar to the ECG acquisition device 10. The ECG acquisition devices 10A-10N are each configured to acquire ECG data; location data; quality data; usage data and identification data as previously described with reference to the ECG acquisition device 10. The ECG acquisition devices 10A-10N may be configured to transmit, either collectively or independently, the ECG data; location data; quality data; usage data and identification data onto the network. For purposes of this disclosure, reference to collective or independent data transmission means the ECG data; location data; quality data; usage data and identification data may be transmitted as a single file, as individual files or any combination thereof.

The ECG device management system 30 may be configured to receive, either collectively or independently, the ECG data; location data; quality data; usage data and identification data from any one of the ECG acquisition devices 10A-10N within any one of the facilities 42A-42N. For purposes of this disclosure, reference to collective or independent data receipt means the ECG data; location data; quality data; usage data and identification data may be extracted from the network 38 as a single file, as individual files or any combination thereof. Advantageously, the ability to selectively extract data allows the management system 30 to receive only the minimum amount of data necessary and thereby most efficiently implement its storage and processing capacities.

The ECG device management system 30 is adapted to facilitate the optimization of the ECG acquisition devices 10A-10N performance. More precisely, the ECG device management system 30 may be configured to provide guidance related to optimal utilization of the ECG acquisition devices. As will be described in more detail, ECG device optimization may relate to advanced capital planning options, pro-actively avoiding workflow disruptions, and finding misplaced or under-utilized systems.

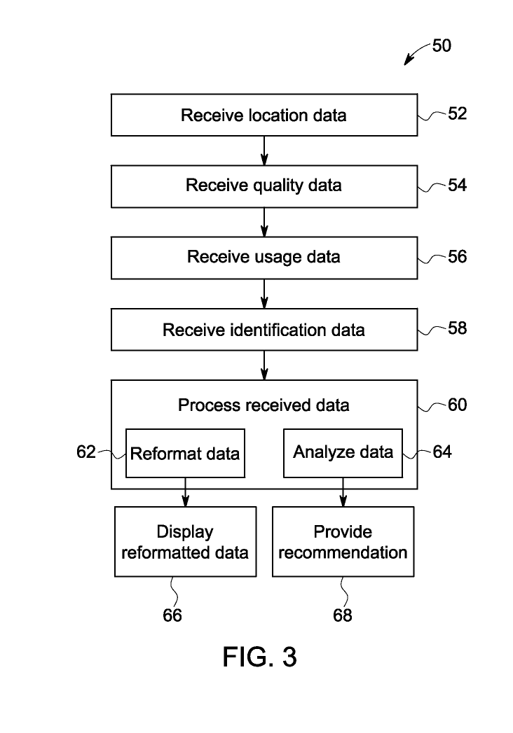

FIG. 3 is a flow diagram representing an exemplary embodiment of the algorithm 50 that may be carried out by the ECG device management system 30. The algorithm 50 may comprise instructions, such as software or code, contained in one or more non-transient computer-readable mediums or persistent storage devices such as the memory 34. The algorithm 50 will hereinafter be described in accordance with an embodiment as comprising steps 52-68. The steps 52-68 need not necessarily be performed in the order shown.

At step 52, the algorithm 50 receives location data from an ECG acquisition device. At step 54, the algorithm 50 receives quality data from an ECG acquisition device. At step 56, the algorithm 50 receives usage data from an ECG acquisition device. At step 58, the algorithm 50 receives identification data from an ECG acquisition device.

At step 60, the algorithm 50 processes the data received at one or more of the steps 52-58. Step 60 will hereinafter be described in a non-limiting manner as comprising either step 62 wherein the algorithm 50 reformats the data, or step 64 wherein the algorithm 50 analyzes the received data.

At step 62 the algorithm 50 reformats the data acquired at one or more of the steps 52-58. This data is preferably reformatted in a manner adapted to highlight opportunities for optimization of the ECG acquisition devices. For example, the data may be graphically depicted over time to in a manner adapted to more clearly convey trends. A trend indicating a consistent decline in usage data may highlight the need to reallocate the associated device(s) to another location. As another example, the data may be graphically depicted over time in a manner adapted to illustrate baseline average values and more clearly convey any significant deviations. A significant deviation may be defined as any deviation correlated with the need for additional scrutiny or follow up action. A significant deviation from established quality data baseline values may highlight the need for service.

At step 66 the reformatted data from step 62 is displayed. The reformatted data may be conveyed via the display 36 of ECG device management system 30 (shown in FIG. 2).

At step 64 the algorithm 50 analyzes the data acquired at one or more of the steps 52-58. Data collected in steps 52-58 may be analyzed at step 64 to identify patterns which contain information that can be used to infer sub-optimal usage or potential malfunction conditions. In addition, the data can be both analyzed and formatted to convey trends that may be also be used for recognition of problem conditions or usage optimization opportunities. The following will provide a non-limiting list of exemplary methods in which the data can be analyzed at step 64. According to one embodiment, the data can be analyzed by assessing whether it exceeds a user a defined range or limit. Examples of user defined ranges or limits include a location range related to a predefined hospital ward or region, a quality limit comprising an ECG file size greater than 35 KB for lossless compression or 17 KB for lossy compression, or a usage limit comprising acquisition and/or transmission of fewer than 5 ECG data files per day.

According to another embodiment, the data analysis of step 64 may initially require training related to the data acquired at steps 52-58. More specifically, the training may comprise the receipt and assessment of large volumes of related data previously acquired under normal operating conditions to establish appropriate baseline average values and learned deviation ranges. According to one embodiment the learned data range is established as the upper and lower limits within which no additional scrutiny or action is required. For example, an analysis of historic usage data may reveal that values between 6 and 30 ECG acquisitions/day have not been correlated with the need for follow up action, and accordingly the learned data range would be 6-30 ECG acquisitions/day. The data analysis of step 64 may then be based on an assessment of whether any newly acquired data remains within a learned deviation range. According to yet another embodiment, user defined thresholds/boundaries may be placed around trained/learned ranges.

At step 68, the algorithm 50 provides a recommendation based on the data analysis of step 64. The recommendation may comprise any known method for communication including but not limited to audible or visual text, alarms, graphic depictions, etc. The recommendation may be conveyed via a local display (e.g., with the display 36), a hardcopy (e.g., with the printer 37) and/or remote electronic notification.

The following will provide a non-limiting list of exemplary recommendations provided at step 68. A first exemplary recommendation may be to find a misplaced acquisition device. This recommendation may be based on data analysis indicating that a given device is outside its range of expected use and is not acquiring its expected volume of ECG data.

Another exemplary recommendation may be to retask an ECG acquisition device. This recommendation may be based on data analysis indicating that a given device is within its range of expected use; the device not acquiring its expected volume of ECG data; and that another disparate group of ECG devices are near their ECG acquisition capacity. The recommendation to retask may alternatively be based on geographic or location volume data indicative of underserved or high-volume areas.

Another exemplary recommendation may be to add additional ECG acquisition devices to increase capacity. This recommendation may be based data analysis indicating that most of the ECG devices within a given facility are near their ECG acquisition capacity. As the percentage of ECG devices within a given facility approach acquisition capacity, the algorithm 50 can provide advanced notice to purchase additional devices for more efficient capital planning.

Another exemplary recommendation may be to initiate service. This recommendation may be based on data analysis indicating that a given device is recording poor quality data, or has monitoring/reporting errors. By generally continuously monitoring quality data the algorithm 50 can provide advanced notice at the early stages of degradation to avoid collection of potentially inaccurate data. Additionally this pro-active service recommendation may avoid or minimize workflow disruptions by allowing for repairs during scheduled downtimes.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An ECG device management system comprising:
a display; and
a processor configured to receive data from a plurality of ECG acquisition devices, wherein the processor is configured to process the data and to generate an output on the display based on the processed data, said output adapted to facilitate the optimization of the ECG acquisition devices performance.

2. The ECG device management system of claim 1, wherein said processor is configured to reformat the data in a manner adapted to highlight opportunities for optimization of the ECG acquisition devices, and wherein generating an output comprises displaying the reformatted data.

3. The ECG device management system of claim 2, wherein said processor is configured to reformat the data by generating a graph adapted to more clearly convey any trends.

4. The ECG device management system of claim 2, wherein said processor is configured to reformat the data by generating a graph adapted to illustrate baseline average values of the data and any significant deviations from the baseline average values.

5. The ECG device management system of claim 1, wherein said processor is configured to analyze the data, and wherein generating an output comprises generating a recommendation related to the ECG acquisition devices.

6. The ECG device management system of claim 5, wherein said processor is configured to analyze the data by assessing whether the data remains within a user defined range.

7. The ECG device management system of claim 5, wherein said processor is configured to analyze the data by assessing whether the data remains within a learned deviation range.

8. The ECG device management system of claim 1, wherein the data comprises location data, quality data, or usage data related to one of the plurality of ECG acquisition devices.

9. An ECG device management system comprising:
a processor configured to receive data from a plurality of ECG acquisition devices;
an algorithm configured to identify patterns within the received data; and
a display operatively connected to the processor, said display configured to visually convey output related to the identified patterns, said output adapted to facilitate the optimization of the ECG acquisition devices performance.

10. The ECG device management system of claim 9, wherein the algorithm is further configured to identify trends based on the received data, and to compute statistical representations of the received data.

11. The ECG device management system of claim 9, further comprising a memory device.

12. The ECG device management system of claim 9, further comprising a printer.

13. A method comprising:
receiving data from a plurality of ECG acquisition devices;
implementing a processor to analyze the data; and
generating a recommendation based on the analyzed data, said recommendation being adapted to facilitate the optimization of the ECG acquisition devices performance.

14. The method of claim 13, wherein said implementing a processor to analyze the data comprises assessing whether the data remains within a user defined range.

15. The method of claim 13, wherein said implementing a processor to analyze the data comprises assessing whether the data remains within a learned deviation range.

16. The method of claim 13, wherein said receiving data comprises receiving location data, quality data, or usage data related to one of the plurality of ECG acquisition devices.

17. The method of claim 13, further comprising implementing a display to visually or audibly convey the recommendation.
